(19) United States
(12) Patent Application Publication
(10) Pub. No.: US 2024/0139259 A1
(43) Pub. Date: May 2, 2024

(54) ENZYME AND PROBIOTICS TAKEN WITH PROTEIN, CREATE A WHOLISTIC SYSTEM THAT PREVENTS THE NEGATIVE EFFECTS OF PROTEIN INDIGESTION ON GUT MICROBIOTA, PROMOTE MICROFLORA BALANCE, AND INCREASE THE BIOAVAILABILITY OF POSTBIOTICS

(71) Applicant: Advanced Enzyme Technologies Ltd., Maharashtra (IN)

(72) Inventors: Abhijit RATHI, Maharashtra (IN); Swati JADHAV, Maharashtra (IN); Vasant Laxminarayan RATHI, Yorba Linda, CA (US)

(73) Assignee: Advanced Enzyme Technologies Ltd., Maharashtra (IN)

(21) Appl. No.: 18/202,091

(22) Filed: May 25, 2023

(30) Foreign Application Priority Data
Oct. 27, 2022 (IN) 202221061309

(51) Int. Cl. A61K 35/742; A61K 35/747; A61K 38/43; A61P 1/14; C12N 9/48; C12N 9/78; C12N 9/88

(52) U.S. Cl. CPC A61K 35/742; A61K 35/747; A61K 38/43; A61P 1/14; C12N 9/485; C12N 9/78; C12N 9/88; C12Y 305/03006; C12Y 401/01011; A61K 2035/115

(57) ABSTRACT

The present invention relates to the use of proteins, enzymes and probiotics to create a wholistic system that prevents negative effects of protein indigestion on gut microbiota and improves diversity and count of gut microbiota, thereby promoting microflora balance. The invention, in one or more embodiments, increases the bioavailability of postbiotics that regulate health, including, skin health, stress, anxiety and depression, physical endurance, healthy aging, inflammation and cardiac health. The invention, in one or more embodiments, also increases the bioavailability of essential, non-essential and branched amino acids.

### CROSS-REFERENCE TO RELATED APPLICATIONS

This disclosure claims priority to Indian Patent Application No. 202221061309 filed on Oct. 27, 2022 and titled “ENZYME AND PROBIOTICS, TAKEN WITH PROTEIN, CREATE A WHOLISTIC SYSTEM THAT PREVENTS THE NEGATIVE EFFECTS OF PROTEIN INDIGESTION ON GUT MICROBIOTA, PROMOTE MICROFLORA BALANCE; AND INCREASE THE BIOAVAILABILITY OF POSTBIOTICS;”, which is incorporated by reference in its entirety.

### TECHNICAL FIELD

The present invention relates to the use of protein, enzymes and probiotics to create a wholistic system that prevents the negative effects of protein indigestion on gut microbiota and improves diversity and count of gut microbiota, thereby promoting microflora balance.

### BACKGROUND

Protein is a key dietary component, vital for human growth, development and health. Protein is comprised of the union of simpler molecules called amino acids, that bind together through peptide bonds. During the digestive process, the human digestive system breaks down protein into amino acids or small peptides using intrinsic digestive proteases like pepsin and pancreatin.

However, inefficient protein digestion regularly occurs because of excess protein intake (e.g., a high protein diet), protease deficiencies or resistance of the protein to digestion. When protein is not appropriately broken down by the digestive system, it can have an overall negative effect on the body, including bloating, stomach cramps, diarrhea increased stress on the digestive system, and a negative impact on gut microbiota. Incomplete digestion of protein can lead to amino acid deficiency, which is associated with nutritional deficiencies, fatigue, accelerated aging, a depressed immune 20 system, weight loss, pressure sores, diarrhea, hair and skin depigmentation, and muscle weakness (Li et al., 2007). Similarly, a high protein diet is known to potentially cause intestinal leakage, colon cancer, and digestive disorders as well as decrease short chain fatty acid production (Marchesi et al., 2016) (Duncan et al., 2007; Salonen et al., 2014). The term “gut microbiota” refers to the group of microorganisms that live in the human gastrointestinal system, as well as their genomes and metabolites. A complex and dynamic community of microorganisms are present in the human gastrointestinal (GI) tract. Both during homeostasis and sickness, these bacteria have a significant impact on the host. Gut microbiota are known to stimulate the immune system, enhance food digestion and absorption, inhibit the growth of pathogenic flora, and preserve the integrity of the intestinal barrier (Zółkiewicz, 2020).

Undigested protein's impact on gut microbiota is well-documented. Incomplete protein digestion changes the ratio of Bacteroidetes to Firmicutes in the gut which is associated with increased tumorigenesis and colorectal cancer. It further leads to the reduction of carbohydrate-utilizing microbiota such as Lachnospiraceae, Ruminococcaceae, Prevotella, Bifidobacterium animalis, Faecalibacterium prausnitzii, and Ruminococcus bromii (Amaretti et al., 2019). A high protein diet taken for a long time has been shown to decrease beneficial organisms in human gut microbiota, especially propionate-producing and butyrate-producing bacteria. A low level of propionate and butyrate in the gut creates a conducive environment for the growth of pathogenic bacteria (Mu et al., 2016; Mu et al., 2017).

The negative impact of protein indigestion on gut microbiota occurs because undigested protein is transported to the colon, where it is fermented by gut microbiota. Excessive fermentation results in the production of amines, hydrogen sulphide, p-cresol and ammonia, which can damage the colon epithelium (Arumugam et al., 2011). Altered gut bacterial composition (dysbiosis) has been associated with the pathogenesis of many inflammatory diseases and infections.

The effect of protein indigestion on gut microbiota has a corresponding effect on postbiotic production and bioavailability. Postbiotics are compounds that support the health of the host, which are produced by beneficial microbiota. Other names for postbiotics include parabiotics, metabiotics, pharmacobiotics, non-viable probiotics, inactivated probiotics, non-biotics, ghost probiotics, or heat-killed probiotics. Postbiotics can modulate human health through different biological activities including, anti-inflammatory, antibacterial, immunomodulatory, anti-carcinogenic, anti-oxidative, antihypertensive, anti-proliferative, and hypocholesterolaemic. Known postbiotics include exopolysaccharides (e.g., uronic acid, kefiran, β-glucans), extracellular enzymes (e.g., glutathione peroxidase, peroxide dismutase, catalase), short chain fatty acids (e.g., acetic, propionic, and butyric acids), vitamins (e.g., B and K), ATP, 5 aromatic amino acids, D-amino acids, non-protein amino acids (e.g., GABA, L-citrulline, L-ornithine, β-alanine), volatile organic compounds, glutathione, antimicrobial peptides (e.g., bacteriocins and defensins), bioactive peptides, dipeptides (e.g., anserine), phytoestrogens (e.g., equol, enterolactone, urolithin A and B), indoles etc. Reduced postbiotics bioavailability can have compounding effects on overall health, including mental health, physical health, cognitive health, skin health, cardiac health, digestive health and immunity.

Therefore, there is need of a method to overcome the negative effects of protein indigestion on gut microbiota, bioavailability of postbiotics without compromising the benefits of protein consumption.

The applicant addresses this need by using the novel composition of enzymes and probiotics with protein.

Advantageously, the applicant found that the combination of enzymes and probiotics improves and balances the gut microbiota even after ingestion of a high protein diet.

Advantageously, the applicant found that the combination of enzymes and probiotics improves gut microbiota as shown by increased numbers of Bacteroides and Lactobacillaceae sp. in the gut after ingestion of protein.

Advantageously, the applicant found that the combination of enzymes and probiotics increases the bioavailability of postbiotics such as GABA, L-citrulline, Lornithine, anserine, β-alanine after ingestion of protein.

Advantageously, the applicant found that the combination of enzymes and probiotics increases the bioavailability of amino acids with ingestion of protein.

### SUMMARY

In one aspect, the present invention relates to a method for preventing the negative effect of protein indigestion on gut microbiota, promoting microflora balance and increasing the bioavailability of postbiotics. The method comprises administration of protein or protein derivatives, enzymes, probiotics and/or one or more other components.

The protein indigestion may occur due to for various reasons, including but not limited to excess protein intake, resistance of the consumed protein to the digestion, digestive enzyme deficiency, or age-related digestive inefficiency.

The protein or protein derivative is derived from plants, animals or microbes.

The gut microbiota is improved in diversity and count of beneficial bacteria, including but not limited to strains belonging to the genus Bacteroides and the genus Lactobacillus.

The probiotics include: Bacillus coagulans, Bacillus clausii, Bacillus subtilis, Lactobacillus acidophilus and/or Lactobacillus plantarum.

The enzymes are proteases, including, endoproteases, exoproteases, carboxyproteases, fungal proteases, bacterial proteases, peptidases, amino proteases and mixtures thereof. The enzymes and protein or protein derivatives are taken simultaneously, or serially wherein the enzymes are taken within 3 hours after the protein is consumed or the enzymes are taken within 1 hour prior to the protein consumption, and the probiotics may be taken at any point within the same day.

The improved bioavailability of postbiotics is measured by a number of metrics, including but not limited to absorption rate, AUC, Cmax and Tmax.

The postbiotics include, but are not limited to, gamma amino butyric acid (GABA), Lcitrulline, β-alanine, L-ornithine and anserine.

The method increases the bioavailability of GABA which plays a crucial role in modulating synaptic transmission, promoting neuronal development and relaxation, preventing sleeplessness and depression, protecting the cardiovascular system, functional antioxidant management pain relief, the prevention of diabetes, as well as in the treatment of depression, insomnia, cognitive impairment, memory loss, mood disorders, seizures, and epilepsy.

The method increases the bioavailability of L-citrulline which plays a crucial role in blood pressure reduction, regulation of innate and adaptive immunity, functional antioxidant management, muscle and metabolic health, cardiac function, age-related muscle damage, endothelial vasodilation, and anti-diabetic functions.

The method increases the bioavailability of β-alanine which plays a crucial role in improving athletic performance, enhanced cognitive function during stressful conditions, improved resiliency to stress, and also increases cellular oxygen consumption and the expression of cellular proteins associated with oxidative metabolism.

The method increases the bioavailability of L-ornithine which plays a crucial role in stress reduction, improved sleep quality, physical fatigue reduction, increasing mental ability, improving skin aesthetics, promoting the synthesis and production of collagen, increasing muscle growth and preventing obesity.

The method increases the bioavailability of anserine which plays a crucial role in fatigue and stress relief, modulation of anxiety, post-partum lactation, improvement of physical performance, immunity, reduction of hyperglycemia and hypertension, enhancing immunity, preventing age related neurological disorders, enhancing the defense against infectious diseases and acceleration of wound healing.

Each serving of probiotics blend include 0.05 billion to 100 billion CFU of Bacillus coagulans, 0.05 billion to 100 billion CFU of Bacillus clausii, 0.05 billion to 100 billion CFU of Bacillus subtilis, 0.05 billion to 100 billion CFU of Lactobacillus acidophilus, and 0.05 billion to 100 billion CFU of Lactobacillus plantarum.

Each serving of one or more enzymes ranges 1 mg to 5 grams in composition. The composition of probiotics and enzymes are consumed at a dose of 0.01 to 10% of the weight of the protein.

In another aspect, the present invention relates to a method for increasing the absorption of amino acids. The method comprises administration of protein or protein derivatives, enzymes and probiotics, wherein the enzymes are selected from one or more of endoproteases, exproteases, carboxyproteases, fungal proteases, bacterial proteases, peptidases, amino proteases, and the probiotics are selected from Bacillus coagulans, Bacillus clausii, Bacillus subtilis, Lactobacillus acidophilus and Lactobacillus plantarum.

The method increases the bioavailability of amino acids, where the amino acids can be essential, non-essential, branched-chain or a mixture thereof.

### BRIEF DESCRIPTION OF THE DRAWINGS

FIG. 1 shows the relative makeup of the gut microbiota by phylum before consuming the whey protein and enzymes-probiotics blend.

FIG. 2 shows the relative makeup of the gut microbiota by phylum after consuming the whey protein and enzymes-probiotics blend for 15 days.

FIG. 3 shows the differential abundance of Bacteroides before and after consuming the protein and enzymes-probiotics blend.

FIG. 4 shows increase in relative abundance of Lactobacillaceae in the gut microbiota of the subjects after consuming protein and enzymes-probiotics blend.

FIG. 5 shows increase in abundance of Weissella in the gut microbiota of the subjects after consuming protein and enzymes-probiotics blend for 15 days.

### DETAILED DESCRIPTION

Aspects of the present invention is directed towards a method for reversing the negative effect of protein indigestion on gut microbiota, promoting microflora balance and increasing the bioavailability of postbiotics. In one or more embodiments, the method comprises administration of protein or protein derivatives, enzymes and probiotics.

The composition of probiotics, enzymes and/or one or more additional components taken along with protein where protein indigestion may occur, including with a high protein diet promotes the rebalance of the intestinal ecosystem to improve the count and diversity of gut microbiota.

In one or more embodiments, the composition of probiotics and enzymes and/or one or more additional component increases the bioavailability of GABA, L-citrulline, s-alanine, L-ornithine, and anserine after ingestion of protein diet.

The composition of probiotics, enzymes and/or one or more additional components taken along with protein may increase the absorption of amino acids.

The World Health Organization defines probiotics as “live microorganisms” which, when administered in adequate amounts, confer a health benefit on the host. Complex colonies of bacteria and other microorganisms occur inside and outside the body of all living beings. This is called the microbiome. Probiotics are live microorganisms, which, when consumed in appropriate amounts, interact synergistically with the microbiome. This in turn positively influences various bodily functions and facilitates better health. Probiotics can transiently colonize the human gut mucosa, influence the intestinal microbiota and exert their effects usually in the gastrointestinal tract.

In an embodiment, the probiotics are selected from but not limited to genera Lactobacillus, Bifidobacterium, Saccharomyces, Enterococcus, Streptococcus, Pediococcus, Leuconostoc, Bacillus, and Escherichia.

In an embodiment, the probiotics can be present in the form of active, inactive, vegetative, or spore forming.

In an embodiment, the probiotics can be a composition containing some or all of Bacillus coagulans, Bacillus clausii, Bacillus subtilis, Lactobacillus acidophilus, and Lactobacillus plantarum.

Bacillus coagulans is a gram-positive, facultative anaerobic, nonpathogenic, spore-forming, lactic acid-producing bacteria. It can promote intestinal digestion. B. coagulans strains can produce various enzymes that facilitate digestion and excretion. It also regulates host symbiotic microbiota and inhibits the growth of pathogenic bacteria.

Bacillus clausiii is a gram-positive, aerobic, alkalophilic, motile, rod shaped, spore-forming bacterium. It restores normal gastrointestinal flora. Various clinical studies suggest that Bacillus clausii improves immune response and reduces intestinal pathogen colonization.

Bacillus subtilis, also known as hay bacillus, and grass bacillus is a gram positive, rod shaped, spore-forming, motile, facultative aerobic and catalase-positive bacterium. B. subtilis prevents the growth of pathogenic bacteria and enhances nutrient assimilation. It is commonly used as an industrial cell factory, for the production of vitamins, inositol, acetoin, hyaluronic acid and other chemicals.

Lactobacillus acidophilus is a gram-positive, rod-shaped, acidophilic. The acid produced in Lactobacillus acidophilus helps in controlling the growth of Candida albicans. L. acidophilus assists with gastrointestinal infections and prevents/treats diarrhea and maintains normal bacteria balance in the lower intestine.

Lactobacillus plantarum also known as Lactobacillus arabinosus is a gram positive, rod-shaped, non-motile, lactic acid bacterium. It is commonly found in human and other mammalian gastrointestinal tracts, saliva, and various food products. L. plantarum is a facultative heterofermentative bacterium that ferments sugars to produce lactic acid, ethanol, acetic acid, carbon dioxide under certain conditions and selective substrates. It also helps in reducing pathogenic bacteria. In an embodiment, the enzymes can be selected from but not limited to endo-proteases, exo-proteases, peptidases, carboxypeptidases, and aminopeptidases.

Proteases refer to a group of enzymes that are efficient executioners of a common chemical reaction: the hydrolysis of peptide bonds presents in protein, resulting in the release of peptides and amino acids. They are also called proteolytic enzymes or proteinases. Proteases, like all enzymes, specifically act on certain chemical reactions efficiently. Each type of protease has its own function. Proteases are found in plants, animals, and microbes and are involved in many aspects of human biology, including for digestive purposes, the remodeling of extracellular matrices and tissues, the induction of physiological immune responses, blood coagulation, immune function, maturation of prohormones, bone formation, programmed cell death and the recycling of cellular proteins that are no longer needed. An effective dose of a protease can be used as a dietary supplement that can increase the rate of protein digestion and absorption. Increasing the rate of postprandial protein digestion may decrease indigestion and facilitate rapid increase of peptide and amino acid levels in the blood.

In an embodiment, proteases can be produced recombinantly or extracted from various sources, including but not limited to plants, animals or microorganisms.

Suitable plant sources of protease can be selected from, but is not limited to, Carica papaya, Ananas comosus, Ficus carica, and Actinidia deliciosa.

Suitable animal sources of protease can be selected from, but is not limited to, bovine or porcine sources.

Suitable microorganism sources of protease can be selected from, but is not limited to, Bacillus, Trichoderma and Aspergillus species.

In an embodiment, the enzymes and protein or protein derivatives are taken simultaneously, or serially wherein the enzymes are taken within 3 hours after the protein is consumed or the enzymes are taken within 1 hour prior to the protein consumption, and the probiotics may be taken at any point within the same day.

In an embodiment, mixtures of probiotics and proteases taken along with protein maintains the balance of phylum Bacteroidetes and Firmicutes, the most dominant gut microbial phyla, representing about 90% of the gut microbiota. The Firmicutes phylum is primarily composed of genera Lactobacillus, Bacillus, Clostridium, Enterococcus, and Ruminicoccus. The Bacteroidetes phylum predominantly composed of genera such as Bacteroides and Prevotella. A higher level of undigested protein leads to increased harmful substances in the gut with an associated higher risk of metabolic diseases. A high protein diet is known to lead to undigested protein in the gut, which subsequently impacts gut microbiota composition and function. It further decreases Lactobacillus and Bifidobacterium microbes, which are known to be beneficial. This leads to many health issues such as digestive disorders, impaired immune function, and mental disorders.

In an embodiment, blends of probiotics and protease increases the genera Bacteroides and Lactobacillus in the gut during/after ingestion of proteins, protein-rich food, peptides, and/or amino acids derived from plants, animals, and/or microbes. The genera Bacteroides and Lactobacillus are associated with the proteolytic activity of the large intestine. Moreover, the genera Lactobacillus are known to protect the intestinal barrier and improve innate immune response and overall health.

In an embodiment, the invention comprises a method for enhancing the bioavailability of gamma-aminobutyric acid (GABA), L-citrulline, β-alanine, L-ornithine, and anserine, the method comprising administering to the subject the combination of probiotics, enzymes and/or one or more other components with protein, protein-rich food, peptides, and amino acids derived from plants, animals, and/or microbes.

Plant sources of protein can be selected from but are not limited to peas, soybeans, kidney beans, rice, quinoa, sunflowers, hemp, cottonseed, camelina, maize, and legumes.

Animal sources of protein can be selected from are but not limited to meat, fish, poultry, eggs, and dairy.

Microbial sources of protein can be selected from but are not limited to bacteria, fungi, yeast, and algae.

In an embodiment, the composition of probiotics, enzymes and/or one or more additional components consumed with protein increases the bioavailability of GABA, L-citrulline, $-alanine, L-ornithine, and anserine in the blood.

Gamma-aminobutyric acid (GABA) is a non-protein inhibitory neurotransmitter in the central nervous system. In the nervous system, GABA is produced from L-glutamic acid and then released into synaptic cleft. Spinach, sweet potato, kale, and broccoli are rich sources of GABA. Other foods that boost GABA production in humans are barley, beans, and peas. Bifidobacterium and Lactobacillus are GABA-producing strains. GABA's primary role is to modulate synaptic transmission, promoting neuronal development and relaxation and preventing sleeplessness and depression. It has a neuroprotective effect and helps in the treatment of depression, insomnia, cognitive impairment, memory loss, and intestinal immunity. GABA also plays a role in cardio-protection, antioxidation, pain-relief, and anti-diabetic functions. Low GABA levels leads to anxiety, depression, insomnia, mood disorders, seizures and epilepsy.

L-citrulline is a neutral, non-proteinogenic alpha-amino acid, and an important component of the urea cycle in the liver and kidney, which removes ammonia from the blood. Leuconostoc dextranicum, Lactobacillus brevis, and Lactobacillus plantarum isolated from apple were able to produce citrulline from arginine by the arginine deiminase (ADI) pathway (Savino 2011). L-citrulline is advantageous in blood pressure reduction, regulation of innate and adaptive immunity, functional antioxidant management, muscle and metabolic health, cardiac function, age-related muscle damage, endothelial vasodilation, and anti-diabetic functions (Allerton 2018).

β-alanine is a non-proteinogenic amino acid which is produced endogenously in the liver or acquired through diet or supplementation (Trexler 2015). β-alanine is also known to be produced from intestinal bacteria, especially Escherichia coli. These bacteria form β-alanine by decarboxylation of aspartic acid using the bacterial enzyme alpha-aspartate decarboxylase (Tiedje 2010 and Eaton 1994). β-alanine has become a popular constituent in many commercially available sports supplements. β-alanine improves athletic performance, enhances cognitive function during stressful conditions, and improves resiliency to stress. β-alanine also increases cellular oxygen consumption and the expression of cellular proteins associated with improved oxidative metabolism.

L-ornithine is a non-proteinogenic amino acid, and an important component of the urea cycle in the liver and kidney, which removes ammonia from the blood. The amino acid arginine is a precursor for the production of ornithine. Lactobacillus from the human gut microbiota produces L-ornithine from arginine and promotes the gut mucosal formation, thus boosting the body's first line of defense (Qi 2019). Escherichia coli and Pseudomonas aeruginosa, utilizes L-glutamate and N-acetyl-L-ornithine for the production of L-ornithine. Bifidobacterium longum KCTC 5734 was able to produce ornithine from sunsilk (cereal-based, ready-to-eat food) which is rich in glutamic acid and arginine (Choi 2017). L-ornithine has been reported to reduce stress, improve sleep quality, reduce physical fatigue, increase mental ability, improve skin aesthetics, promote the synthesis and production of collagen and effectively increase muscle growth and prevent obesity by enhancing basal metabolism (Miyake 2014 and Yeong 2020).

Anserine (beta-alanyl-3-methyl-L-histidine) is a histidine containing dipeptide that acts as a biochemical buffer, chelator, antioxidant, anti-inflammatory, and anti-glycation agent. Anserine can relieve stress and fatigue, ameliorate anxiety, promote post-partum lactation, improve physical capacity, and exercise performance, reduce hyperglycemia and hypertension, enhance immunity, prevent aging-associated neurological dysfunction and inflammation, enhance defense against infectious diseases and accelerate wound healing.

In an embodiment, the composition of probiotics, enzymes and/or one or more additional components with protein or protein derivatives increases the bioavailability of GABA, L-citrulline, β-alanine, L-ornithine and anserine in the blood.

The parameters used in the study to examine the bioavailability of postbiotics in the blood are measured by a number of metrics, including but not limited to absorption rate, AUC, Cmax and Tmax.

In one embodiment, the composition of probiotics and enzymes and/or one or more additional components with protein or protein derivatives increases the absorption rate of said at least one postbiotic is from 10% to 600%. The absorption rate is measured as the rate at which postbiotics get absorbed in the blood after ingestion of the protein.

In one embodiment, the composition of probiotics and enzymes and/or one or more additional components taken with protein or protein derivatives increases the total absorption rate (expressed as AUC) of at least one postbiotic at a rate between 1% to 100%. AUC is the area under the concentration/time curve measured using the linear trapezoidal rule and all available time points.

In one embodiment, the composition of probiotics and enzymes and/or one or more additional components taken with protein or protein derivatives increases the maximum concentration (expressed as Cmax) of at least one postbiotic in the blood at any particular time point at a rate between 1% to 100%. Cmax is defined as the highest observed concentration of the compound in the blood at any point in time.

In one embodiment, the composition of probiotics and enzymes and/or one or more additional components taken with protein or protein derivatives shows faster bioavailability of at least one postbiotic, as measured by the decrease in the time to reach the Cmax (expressed in Tmax) of the postbiotic, at a rate between 25% to 100%. Tmax is time at which maximum concentration (Cmax) is observed.

In an embodiment, the invention comprises a method for increasing the absorption of amino acids, enhancing the bioavailability of amino-acids, the method comprising administration of protein or protein derivatives, enzymes and probiotics, and one or more additional components wherein the enzymes are selected from one or more of endoproteases, exproteases, carboxyproteases, fungal proteases, bacterial proteases, peptidases, amino proteases, and the probiotics are selected from Bacillus coagulans, Bacillus clausii, Bacillus subtilis, Lactobacillus acidophilus and Lactobacillus plantarum.

Amino acids can be essential, non-essential, branched or a mixture thereof.

Essential amino acids include, but are not limited to, histidine, isoleucine, leucine, lysine, methionine, phenylalanine, threonine, tryptophan, and valine. Non-essential amino acids include, but are not limited to, alanine, arginine, asparagine, aspartic acid, cysteine, glutamic acid, glutamine, glycine, proline serine, tyrosine. Branched-chain amino acids include, but are not limited to, leucine, isoleucine and valine.

Amino acids are involved in many important roles in the body. Amino acids play crucial role in energy balance, lipid and glucose metabolism, immunomodulation, mitochondrial biogenesis, cellular growth, nucleotide and protein synthesis, wound healing, athletic performance, and muscle repair.

In one embodiment, the composition of probiotics and enzymes and/or one or more additional components taken with protein or protein derivatives increases the absorption rate of at least one amino acid at a rate between 1% to 800%.

In one embodiment, the composition of probiotics and enzymes and/or one or more additional components taken with protein or protein derivatives increases the total absorption of at least one amino acid (expressed as AUC) at a rate between 1% to 25%.

In one embodiment, the composition of probiotics and enzymes and/or one or more additional components taken with protein or protein derivatives increases the maximum concentration (expressed as Cmax) of at least one amino acid in the blood at any point in time at a rate between 1% to 30%.

In one embodiment, the composition of probiotics and enzymes and/or one or more additional components taken with protein or protein derivatives shows faster bioavailability of at least one postbiotic, as measured by the decrease in the time to reach the Cmax (expressed in Tmax) of the postbiotic, at a rate between 1% to 50%. Tmax is the time at which the maximum concentration (Cmax) is observed.

### EXAMPLES

Examples are set forth herein below and are illustrative of different amounts and types of reactants and reaction conditions that can be utilized in practicing one or more embodiments.

### Example 1: Combination of Enzymes and Probiotics Taken with Protein Prevents the Negative Effects of Protein Indigestion on Gut Microbiota

Aim

The purpose of the study was to evaluate the effect of enzymes and probiotics consumed 10 with protein (whey or pea proteins) on the gut microbiota.

Subject Population

Thirty (30) healthy Indian male subjects (n=15 in whey; n=15 in pea protein study) between the ages of 18 and 35 years old with a body mass index (BMI) between 19-24.99 kg/m2 were selected for the trial. Subject selection was done on the following inclusion and exclusion criteria:

Inclusion Criteria

Inclusion criteria were: a willingness to provide written informed consent and comply with study instructions for its duration, and normal health as determined by medical history, physical examination, and screening laboratory values within normal limits. Participants who met the necessary inclusion criteria were further encouraged not to change their current physical activity levels and to refrain from exercise for 24 h before starting the clinical trial.

Exclusion Criteria

Exclusion criteria were: subjects with organ transplantation or surgery in the past 6 months; known hypersensitivity or idiosyncratic reaction or intolerance to enzyme/probiotics/protein or any ingredients of the formulation or any related products as well as severe hypersensitivity reactions (like angioedema) to any drugs or food products; women who are pregnant or lactating; a history of smoking or tobacco consumption; a history of clinically significant, cardiovascular, pulmonary, hepatic, renal, hematological, gastrointestinal, endocrine, immunologic, dermatologic, musculoskeletal, neurological or psychiatric disease; a difficulty with donating blood.

Medical History

A medical history was checked at the screening visit to collect information on past and current medical conditions, surgical history, allergy information, and concomitant or recently taken medications.

Physical Examination

A physical examination includes general appearance, skin, head, neck, ENT, heart, lungs, abdomen, extremities, neurological, musculoskeletal, and lymph nodes. Vital signs included but were not limited to pulse rate, respiratory rate, systolic and diastolic pressure, and body temperature.

Study Material

Protein: Whey and pea protein.

Investigational product (IP): Enzymes-probiotics blend, where the enzymes are comprised of various proteases and the probiotics are comprised of Bacillus coagulans, Bacillus clausii, Bacillus subtilis, Lactobacillus acidophilus and Lactobacillus plantarum.

Placebo: Maltodextrin

The materials to be administered, listed above, were supplied in a sealed sachet (Test: protein and enzymes-probiotics blend; Placebo: protein and maltodextrin). Subjects were instructed to open the sachets and mix the contents (Test or Placebo) along with 300-500 ml of lukewarm water and consume on an empty stomach in the morning every day.

Informed Consent

The study was conducted in conformity with ICH-GCP (E6 R2) guidelines, the Helsinki Declaration, and the local regulatory requirements (Indian GCP, Indian Council of Medical Research, and New Drugs and Clinical Trials Rules-2019). The approved protocol was followed with no further changes or amendments during the trial. All subjects provided complete information about the study in written, visual and oral form in an understandable language. Every subject had given digital informed consent to the investigator after understanding the objective of this trial, including possible risks and benefits.

Trial Design

Two separate randomized, double-blinded, placebo controlled, and crossover studies were designed on whey (animal-derived protein) and pea protein (plant-derived protein).

Study duration: Two-day screening and baseline-testing phases followed by two supplementation phases that each spanned 15 days each and were separated by a washout period of 30 days.

Total 30 (n=15 for whey and n=15 for pea) male subjects were randomly assigned to ingest a supplement containing a 30 g of protein (whey or pea) and 1% of placebo (maltodextrin), or a 30 g of protein (whey or pea) and 1% enzymes-probiotics blend for 15 days prior to the experimental test. The participants, the investigators, and the study team were blinded to the treatment allocation. Daily diet was recorded throughout the study.

Sample Analysis

Faecal samples of all the test subjects were collected on Day 1 and Day 15 (of both supplementation phases) and were analyzed for gut microbiota. Gut microbiota of all the test subjects were analyzed by the shotgun metagenome sequencing method. Stool samples were collected from all participants using the Invitek Molecular Stool Collection Module [Cat. No. 1038111300, Berlin, Invitek Molecular GmbH]. Each participant was given the stool collection kit, with clear instructions about sample collection. The stool collection tube contained 8 ml of DNA stabilizing solution and an integrated spoon in cap. All participants were instructed to collect ˜4-5 spoons of stool into the 8 ml stabilizing solution. Once collected, they were instructed to gently mix the sample with the stabilizing solution for 15 seconds, seal and then ship the sample under room temperature to the processing unit for DNA extraction. DNA was extracted from stool samples using QIAamp® Fast DNA Stool Mini (Cat No./ID: 51604, QIAGEN) following the manufacturer's “Fast DNA Stool Mini Handbook” for fast purification of genomic DNA. Whole metagenome sequencing was performed on all samples using long read sequencing technology. Briefly, the DNA library was prepared with the Ligation sequencing kit (SQK-LSK109) (Oxford Nanopore Technologies (ONT), Oxford, UK), then loaded onto a R9.4.1 MinION flow cell (FLO-MIN106) and sequenced on the ONT MinION Mk1B device (MIN-101B). Basecalling and demultiplexing of sequence reads was performed with Guppy v4.2.2 and with assistance by MinKNOW GUI v4.1.22. Raw sequencing reads were stored in FastQ format for further computational analysis. Change in gut microbiome profiles in the subject before and after consumption of whey/pea protein with enzymes-probiotics blend for 15 days was characterized and visualized for direct quantitative comparison of abundances, and differentially abundant species across the comparing groups.

Results

The results indicate that gut microbiota is improved by restoring the bacteroidetes to firmicutes ratio, increasing proteolytic species, and the genera Bacteroides and Lactobacillus in the gut. At the phylum level, an increase in the abundance of Bacteroidetes, and a decrease in the abundance of Firmicutes and Actinobacteria was observed after consumption of whey protein with the enzymes-probiotics blend. The increase in Bacteroidetes was in turn due to an increase in the abundance of several species of Phocaeicola (Phocaeicola plebeius and Phocaeicola coprocola) and Prevotella (Prevotella copri and Prevotella hominis). While the decrease in Firmicutes was largely due to reduction in relative abundance of Streptococcus lutetiensis and Streptococcus equinus, the reduction in Actinobacteria was due to the reduced abundance of Atopobium sp. In a healthy individual, in terms of average abundance of organisms, 4 phyla have abundance above 1%, these are Actinobacteria (1.82±3%), Bacteroidetes (73.13±22.16%), Firmicutes (22.2±18.66%) and Proteobacteria (2.15 10±10.39%). Bacteroids sp. was found to be increased in the subjects after consumption of whey protein with the enzymes-probiotics blend. These species in the gut microbiota are proteolytic organisms. Increased Bacteroids sp is associated with proteolytic organisms for protein degradation.

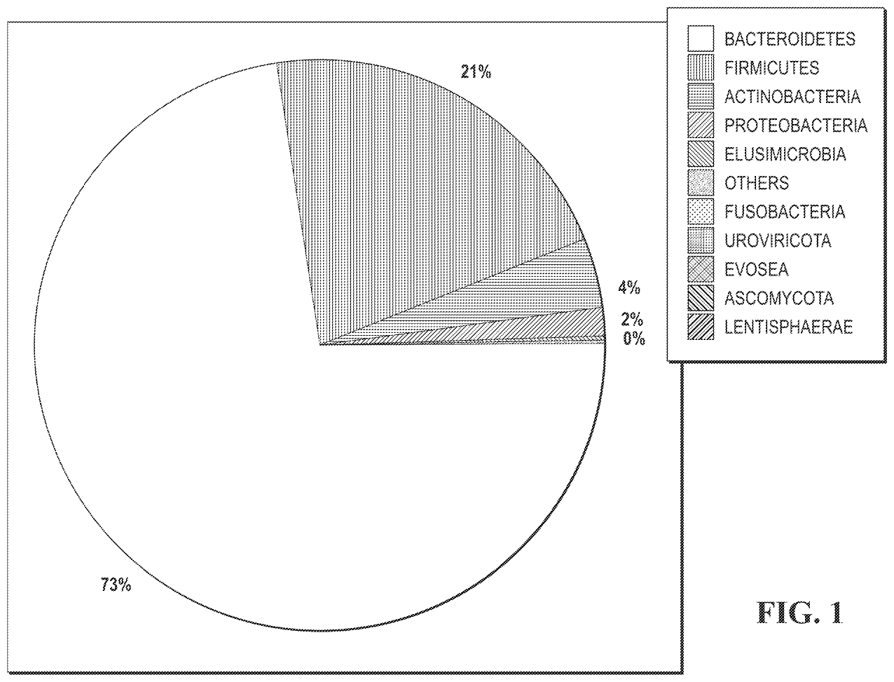

FIG. 1-3 reports gut microbiota changes in the subjects before and after consuming whey protein with the enzymes-probiotics blend for 15 days.

FIG. 1 reports the relative makeup of the gut microbiota by phylum before consuming the whey protein and enzymes-probiotics blend.

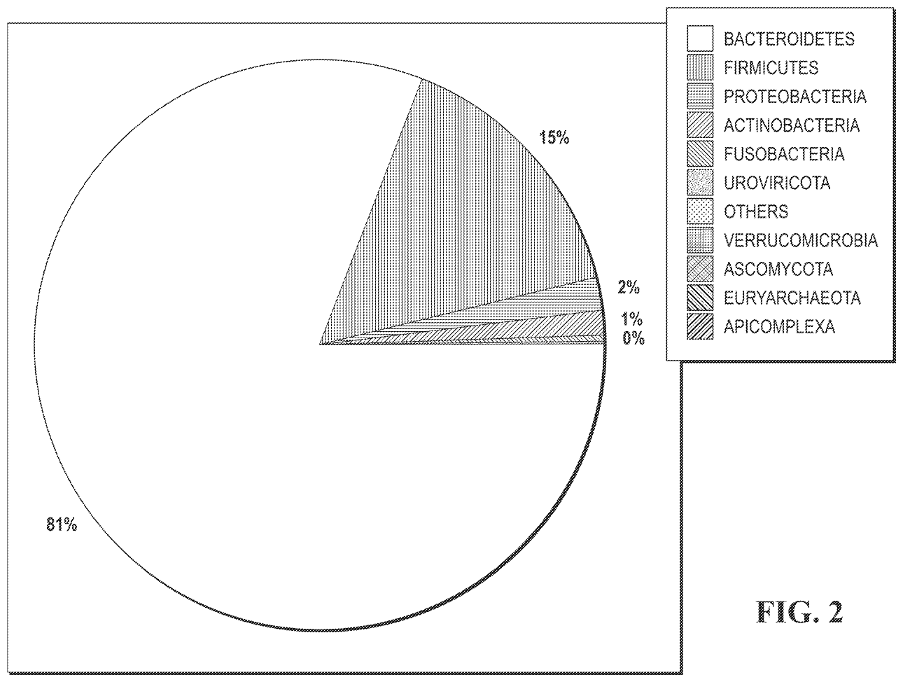

FIG. 2 reports the relative makeup of the gut microbiota by phylum after consuming the whey protein and enzymes-probiotics blend for 15 days.

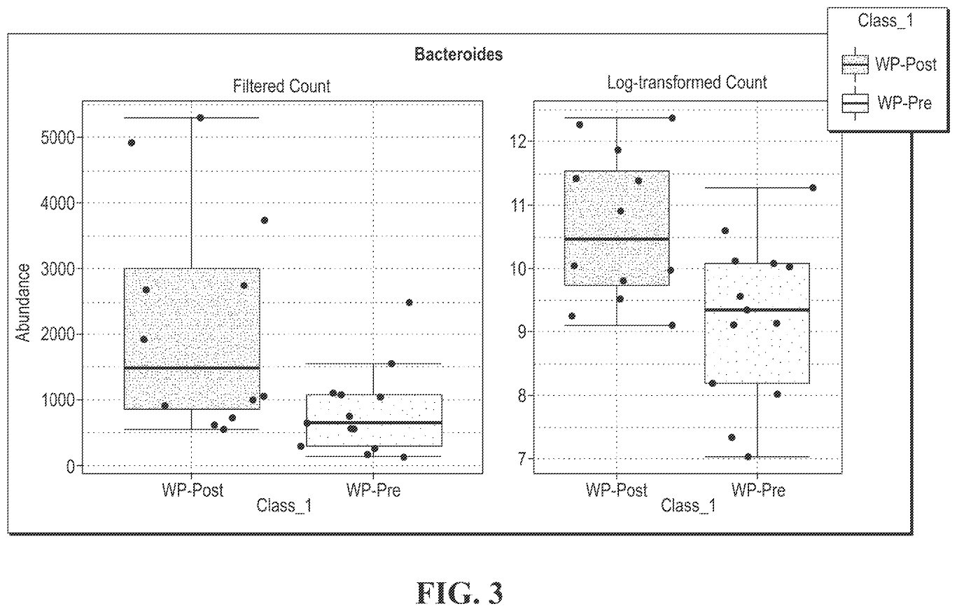

FIG. 3 reports the differential abundance of bacteroides before and after consuming the protein and enzymes-probiotics blend.

### Example 2: Enzymes and Probiotic Blend with Protein Promotes Postbiotic-Producing Gut Microbiota

Aim

Purpose of the study was to evaluate the effect of enzymes and probiotics blend consumed along with the protein (animal or plant derived proteins) on the gut microbiota.

Study Design and Sample Analysis

Study design was similar to the Example 1. Fecal samples of all the test subjects were collected on Day 1 and Day 15 (of both the supplementation period) and were analyzed for gut microbiota using a same method as explained in Example 1.

Results

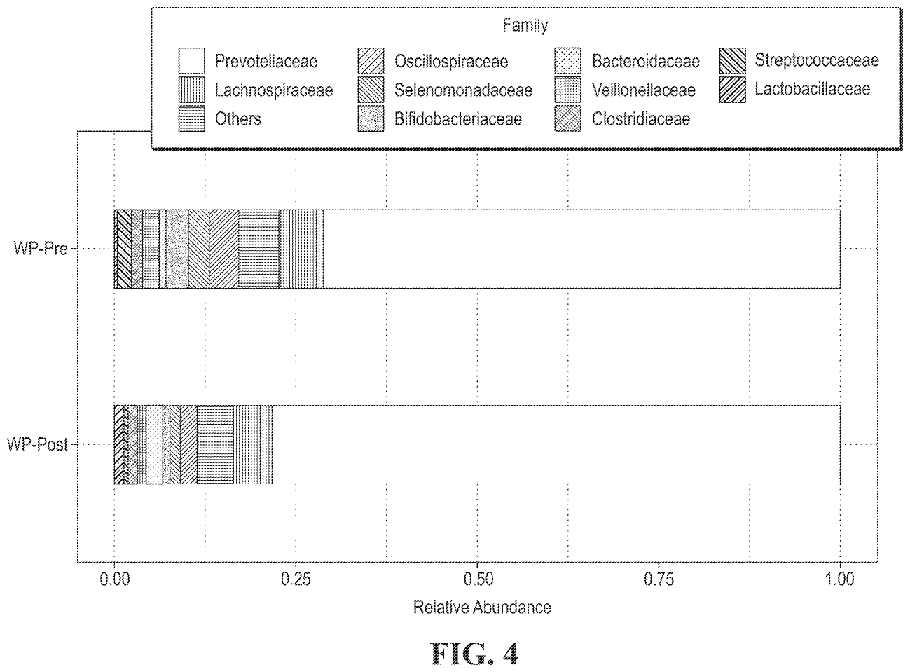

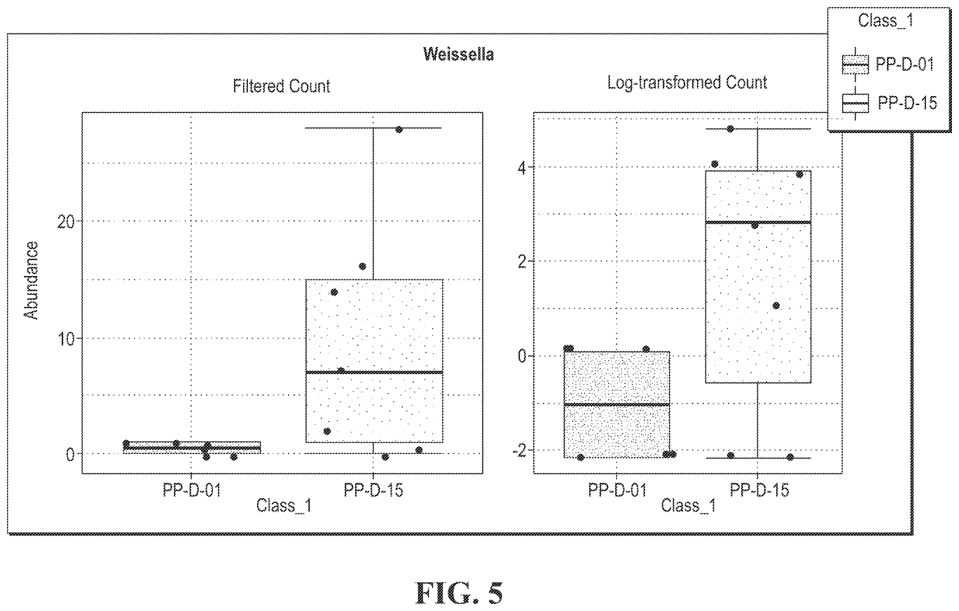

FIG. 4-5 reports the changes in the gut microbiota of the subjects before and after 10 consuming protein with enzymes-probiotics blend for 15 days.

FIG. 4 reports increase in relative abundance of Lactobacillaceae in the gut microbiota of the subjects after consuming protein and enzymes-probiotics blend.

FIG. 5 reports increase in abundance of Weissella in the gut microbiota of the subjects after consuming protein and enzymes-probiotics blend for 15 days.

Results indicated that relative abundance of the Lactobacillaceae family was increased after consumption of protein with enzymes-probiotics blend for 15 days. This family is commonly associated with the production of different postbiotics. Weissella is a genus of gram-positive bacteria placed within the family Lactobacillaceae. Results showed increased abundance of Weissella after consumption of protein along with the enzymes probiotics blend. Supplementation of enzymes-probiotics blend along with the protein is effective in promoting postbiotics producing gut microbiota.

### Example 3: Enzymes and Probiotic Blend Taken Alone with the Animal Derived Protein (Whey) Increases the Bioavailability of the Postbiotics in the Blood

Aim

Purpose of the study was to evaluate the effect of enzymes and probiotics blend taken along with whey protein on the subsequent appearance of postbiotics in the blood.

Study Design and Sample Analysis

Study design is same as explained in example 1. On day 15 of the study, site visit was planned. On arrival to site, after the baseline assessments, subjects were administrated with respective supplements on empty stomach and blood samples were withdrawn at 0 h, 0.5 h, 1 h, 2 h, 3 h and 4 h for postbiotics analysis. Postbiotics were analyzed using liquid chromatography tandem mass spectrometry method.

Results Variables

Amount of postbiotics (non-protein amino acid) (i.e. ornithine, beta-alanine, anserine, citrulline, GABA) in peripheral blood measured by means of: maximum concentration (Cₘₐₓ); the corresponding time (Tₘₐₓ) and area under the curve (AUC), rate of absorption of non-protein amino acid in μmol/min and increase (%) calculated as appropriate.

The area under the curve (concentration vs. time, in short AUC) was calculated for the non-protein amino acids through the linear trapezoidal rule and using all available time points. Cₘₐₓ was defined as the highest observed concentration and Tₘₐₓ was the time when Cₘₐₓ was reached. Increase (%) in AUC, increase (%) in Cₘₐₓ and decrease (%) in Tₘₐₓ was calculated for test group compared to placebo.

Results

Results reveled subjects who consumed whey protein plus enzymes-probiotics blend (test arm) showed increased rate of absorption of postbiotics compared to the subjects who consumed only whey protein (placebo arm). In particular, rate of absorption of ornithine (362%), citrulline (374%), GABA (208%), and beta-alanine (540%) was increased in the subjects who consumed enzymes-probiotics blend along with the whey protein.

Results further indicated increase (%) in AUC and Cₘₐₓ of anserine (AUC 70.1%, Cₘₐₓ 74%) and citrulline (AUC 7.6%, Cₘₐₓ 7.6%) in the subjects who consumed enzymesprobiotics blend along with the whey protein. Also decrease (%) in Tₘₐₓ was observed for ornithine (75%), beta-alanine (50%) and anserine (50%) in subjects who consumed enzymes-probiotics blend along with the whey protein.

Tables 1-3 reports the results for the participant (n=15) consuming animal derived protein (whey) with and without enzymes-probiotics blend in a randomized, cross-over double blind clinical trial.

Table 1 reports the rate of absorption of postbiotics in the subjects consuming whey protein and the subjects consuming whey protein with enzymes-probiotics blend.

Table 2 reports the AUC and Cₘₐₓ of postbiotics in the subjects consuming whey protein and the subjects consuming whey protein with enzymes-probiotics blend.

Table 3 reports Tₘₐₓ of postbiotics in the subjects consuming whey protein and the subjects consuming whey protein with enzymes-probiotics blend.

TABLE 1
Rate of absorption of postbiotics in the subjects
consuming whey protein and the subjects consuming
whey protein with enzymes-probiotics blend.

| | Rate of absorption of non-protein | | |
|---|---|---|---|
| | amino acid (μmol/min) | | |
| | | Whey Protein + | |
| | Whey | Enzymes-Probiotics | Increase |
| Postbiotics | Protein | Blend | % |
| Ornithine | 0.103 | 0.475 | 362 |
| Citrulline | 0.025 | 0.12 | 374 |
| Gamma-aminobutyric acid | 0.014 | 0.042 | 208 |
| Beta- alanine | 0.001 | 0.008 | 540 |

TABLE 2
AUC and Cmax of postbiotics in the subjects consuming whey protein and
the subjects consuming whey protein with enzymes-probiotics blend.

| | | | Whey Protein + | | | |
|---|---|---|---|---|---|---|
| | | | Enzymes-Probiotics | | | |
| | Whey Protein | | Blend | | | |
| | AUC | Cₘₐₓ | AUC | Cmax | Increase (%) | |
| Postbiotics | (μmol*h/L) | (μmol/L) | (μmol*h/L) | (μmol/L) | AUC | Cₘₐₓ |
| Anserine | 0.747 | 0.196 | 1.27 | 0.341 | 70.1 | 74 |
| Citrulline | 198 | 56 | 213 | 61 | 7.6 | 7.6 |

TABLE 3
Tₘₐₓ of Postbiotics in the subjects consuming whey protein and the
subjects consuming whey protein with enzymes-probiotics blend.

| Tₘₐₓ (hr.) | | | |
|---|---|---|---|
| | | Whey Protein + | |
| | Whey | Enzymes-Probiotics | Decrease |
| Postbiotics | Protein | Blend | (%) |
| Ornithine | 2 | 0.5 | 75 |
| Beta- alanine | 1 | .05 | 50 |
| Anserine | 2 | 1 | 50 |

### Example 4: Enzymes and Probiotic Blend Taken Alone with the Plant Derived Protein (Pea) Increases the Bioavailability of the Postbiotics in Blood

Aim

Purpose of the study was to evaluate the effect of enzymes and probiotics blend taken along with pea protein on the subsequent appearance of postbiotics (non-protein amino acid) in the blood.

Study Design and Sample Analysis

Study design was same as explained in Example 1 and sample analysis was same as mentioned in Example 3.

Results Variables

Same as explained in Example 3.

Results

Results reveled subjects who consumed pea protein plus enzymes-probiotics blend (test arm) showed increased rate of absorption of postbiotics compared to the subjects who consumed only pea protein (placebo arm). In particular, rate of absorption of beta-alanine (15%) was increased in the subjects who consumed enzymes-probiotics blend along with the whey protein.

Results further indicated increase (%) in AUC and Cmax of anserine (AUC 6.7%, Cmax 15.1%) and citrulline (AUC 10.4%, Cmax 12.9%) in the subjects who consumed enzymes-probiotics blend along with the pea protein. Also decrease (%) in Tmax was observed for GABA (67%) in subjects who consumed enzymes-probiotics blend along with the whey protein.

Tables 4-6 reports the results for the participant (n=15) consuming plant origin protein (pea) with and without enzymes-probiotics blend in a randomized, cross-over double blind clinical trial.

Table 4 reports the rate of absorption of postbiotics in the subjects consuming pea protein and the subjects consuming pea protein with enzymes-probiotics blend.

Table 5 reports the AUC and Cₘₐₓ of postbiotics in the subjects consuming pea protein and the subjects consuming pea protein with enzymes-probiotics blend.

Table 6 reports Tₘₐₓ of postbiotics in the subjects consuming pea protein and the subjects consuming pea protein with enzymes-probiotics blend.

TABLE 4
Rate of absorption of postbiotics in the subjects
consuming pea protein and the subjects consuming
pea protein with enzymes-probiotics blend.

| | Rate of absorption of non- protein amino | | |
|---|---|---|---|
| | acid (μmol/min) | | |
| | | Pea Protein + | |
| | Pea | Enzymes-Probiotics | Increase |
| Postbiotics | Protein | Blend | (%) |
| β- alanine | 0.002 | 0.003 | 15 |

TABLE 5
AUC and Cₘₐₓ of postbiotics in the subjects consuming pea protein and
the subjects consuming pea protein with enzymes-probiotics blend.

| | | | Pea Protein + Enzymes- | | | |
|---|---|---|---|---|---|---|
| | Pea Protein | | Probiotics Blend | | | |
| | AUC | Cₘₐₓ | AUC | Cmax | Increase | |
| Postbiotics | (μmol*h/L) | (μmol/L) | (μmol*h/L) | (μmol/L) | AUC | Cₘₐₓ |
| Anserine | 0.975 | 0.257 | 1.041 | 0.295 | 6.7 | 15.1 |
| Citrulline | 277 | 72 | 305 | 81 | 10.4 | 12.9 |

TABLE 6
Tₘₐₓ of postbiotics in the subjects consuming pea protein and the
subjects consuming pea protein with enzymes-probiotics blend.

| | Tₘₐₓ (hr.) | | |
|---|---|---|---|
| | | Pea Protein + | |
| | Pea | Enzymes-Probiotics | Decrease |
| Postbiotics | Protein | Blend | (%) |
| Gamma-amniobutyric | 3 | 1 | 67 |
| acid | | | |

### Example 5: Enzymes and Probiotic Blend Taken Alone with Animal Derived Protein (Whey) Increases the Bioavailability of Amino Acids in the Blood

Aim

Purpose of the study was to evaluate the effect of enzymes and probiotics blend consumed along with whey protein (animal derived protein) on the subsequent appearance of amino acids in peripheral blood.

Study Design and Sample Analysis

Study design is same as explained in example 1. On day of the study site visit was planned. On arrival to site, after the baseline assessments, subjects were administrated with respective supplements on empty stomach and blood samples were withdrawn at 0 h, 0.5 h, 1 h, 2 h, 3 h and 4 h for amino acid analysis. Amino acids were analyzed using liquid chromatography tandem mass spectrometry method.

Results Variables

Amount of amino acids (i.e. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine) in peripheral blood were described by means of: area under the curve (AUC), maximum concentration (Cmax), the corresponding time (Tmax) and, and rate of increase in percentage as well as μmol/min, calculated as appropriate.

The area under the curve (concentration vs. time, in short AUC) was calculated for each of the 20 amino acids through the linear trapezoidal rule and using all available time points. Cmax was defined as the highest observed concentration and Tmax was the 10 time when Cmax was reached. Increase (%) in AUC, increase (%) Cmax and decrease (%) in Tmax was calculated for test group compared to placebo.

Results

Results revealed that the individuals who took whey protein plus enzymes-probiotics blend (test arm) showed increase in AUC and Cmax of arginine (AUC 22.3%, Cmax 15 15.7%), asparagine (AUC 20.6%, Cmax 19.2%), glutamic acid (AUC 4.7%, Cmax 7%), isoleucine (AUC 20.9%, Cmax 27.6%), leucine (AUC 6.5%, Cmax 5.5%), lysine (AUC 16.0%, Cmax 13.8%), methionine (AUC 16.1%, Cmax 18.6%), phenylalanine (AUC 5.3%, Cmax 5.4%), serine (AUC 13.9%, Cmax 15.2%), threonine (AUC 18.56%, Cmax 16.23%), and tryptophan (AUC 12.10%, Cmax 11.67%) compared to the placebo arm.

Also decrease (%) in Tmax was observed for alanine (10%), arginine (12%), asparagine (37%), cysteine (33%), glutamine (12%), glutamic acid (25%), glycine (48%), isoleucine (20%), leucine (31%), lysine (12%), methionine (46%), phenylalanine (24%), proline (24%), serine (2%), tryptophan (16%), tyrosine (24%) and valine (36%).

Results indicated that the rate of absorption of plasma amino acid concentration (μmol/min) in test arm was increased compared to that of in the placebo arm. In particular, alanine (168%), asparagine (41%), aspartic acid (227%), cysteine (117%), glutamine (160%), glutamic acid (57%), isoleucine (100%), leucine (161%), lysine (91%), methionine (116%), phenylalanine (174%), proline (777%), serine (171%), threonine (101%), tryptophan (68%), tyrosine (154%) and valine (51%), which includes both essential amino acids and branched-5 chain amino acids.

Tables

Tables 7-9 reports the results for the participant (n=15) consuming animal derived protein (whey) with and without enzymes-probiotics blend in a randomized, cross-over double blind clinical trial.

Table 7 reports the rate of absorption of amino acid in the subjects consuming whey protein and the subjects consuming whey protein with enzymes-probiotics blend.

Table 8 reports the AUC and Cₘₐₓ of amino acid in the subjects consuming whey protein and the subjects consuming whey protein with enzymes-probiotics blend.

Table 9 reports Tₘₐₓ of amino acid in the subjects consuming whey protein and the subjects consuming whey protein with enzymes-probiotics blend.

TABLE 7
Rate of absorption of plasma amino acid in the subjects
consuming whey protein and the subjects consuming
whey protein with enzymes-probiotics blend.

| | Rate of absorption of plasma amino acid | | |
|---|---|---|---|
| | (μmol/min) | | |
| | Whey | Whey Protein + Enzymes- | Increase |
| Amino acids | Protein | Probiotics Blend | (%) |
| Alanine | 0.85 | 2.27 | 168 |
| Asparagine | 0.51 | 0.72 | 41 |
| Asparatic acid | 0 | 0.001 | 227 |
| Cysteine | 0.08 | 0.16 | 117 |
| Glutamic acid | 0.09 | 0.15 | 57 |
| Glutamine | 1.59 | 4.15 | 160 |
| Isoleucine | 1.25 | 2.51 | 100 |
| Leucine | 1.19 | 3.1 | 161 |
| Lysine | 1.44 | 2.75 | 91 |
| Methionine | 0.19 | 0.4 | 116 |
| Phenylalanine | 0.11 | 0.29 | 174 |
| Proline | 0.18 | 1.54 | 777 |
| Serine | 0.22 | 0.59 | 171 |
| Threonine | 0.58 | 1.17 | 101 |
| Tryptophan | 0.33 | 0.56 | 68 |
| Tyrosine | 0.19 | 0.49 | 154 |
| Valine | 1.51 | 2.28 | 51 |

TABLE 8
AUC and Cₘₐₓ of plasma amino acid in the subjects
consuming whey protein and the subjects consuming
whey protein with enzymes-probiotics blend.

| | | | Whey Protein + | | | |
|---|---|---|---|---|---|---|
| | | | Enzymes-Probiotics | | | |
| Amino | Whey Protein | | Blend | | Increase | |
| acids | AUC | Cₘₐₓ | AUC | Cₘₐₓ | AUC | Cₘₐₓ |
| Alanine | 2621.9 | 759.1 | 2577.3 | 934.8 | −1.7 | 23.1 |
| Arginine | 244.9 | 81.7 | 299.5 | 94.6 | 22.3 | 15.7 |
| Asparagine | 480.7 | 149.3 | 579.7 | 177.9 | 20.6 | 19.2 |
| Aspartic | 4.1 | 1.3 | 4.1 | 1.3 | 0.0 | −2.5 |
| acid | | | | | | |
| Cystine | 146.7 | 47.2 | 131.9 | 41.6 | −10.0 | −11.8 |
| Glutamine | 208.1 | 69.6 | 211.5 | 66.5 | 1.7 | −4.5 |
| Glutamic | 3548.9 | 1014.8 | 3714.0 | 1086.1 | 4.7 | 7.0 |
| acid | | | | | | |
| Glycine | 1282.3 | 376.9 | 1237.7 | 373.3 | −3.5 | −0.9 |
| Histidine | 630.5 | 465.0 | 625.7 | 342.9 | −0.8 | −26.3 |
| Isoleucine | 606.1 | 208.7 | 732.9 | 266.2 | 20.9 | 27.6 |
| Leucine | 879.7 | 308.7 | 936.8 | 325.8 | 6.5 | 5.5 |
| Lysine | 850.6 | 287.1 | 987.0 | 326.8 | 16.0 | 13.8 |
| Methionine | 107.3 | 35.2 | 125.6 | 41.8 | 17.1 | 18.6 |
| Phenylalnine | 236.1 | 67.8 | 248.7 | 71.5 | 5.3 | 5.4 |
| Proline | 1533.0 | 450.4 | 1544.5 | 449.2 | 0.8 | −0.3 |
| Serine | 378.1 | 110.5 | 430.5 | 127.3 | 13.9 | 15.2 |
| Threonine | 629.0 | 187.9 | 745.7 | 218.4 | 18.6 | 16.2 |
| Tryptophan | 305.9 | 94.4 | 343.1 | 105.4 | 12.1 | 11.7 |
| Tyrosine | 290.6 | 90.5 | 283.3 | 85.8 | −2.5 | −5.2 |
| Valine | 1144.7 | 374.2 | 1007.7 | 299.9 | −12.0 | −19.9 |

TABLE 9
Tₘₐₓ of plasma amino acid in the subjects consuming
whey protein and the subjects consuming whey
protein with enzymes-probiotics blend.

Tmax

| | |
|---|---|
| Amino acids | Whey Protein + |

| | Whey | Enzymes-Probiotics | Decrease |
|---|---|---|---|
| | Protein | Blend | (%) |
| Alanine | 1.9 | 1.7 | 10 |
| Arginine | 1.7 | 1.5 | 12 |
| Asparagine | 1.6 | 1.0 | 37 |
| Aspartic acid | 1.8 | 2.0 | −13 |
| Cysteine | 2.1 | 1.4 | 33 |
| Glutamine | 1.4 | 1.2 | 12 |
| Glutamic acid | 1.6 | 1.2 | 25 |
| Glycine | 1.1 | 0.6 | 48 |
| Histidine | 1.7 | 1.7 | −2 |
| Isoleucine | 1.3 | 1.1 | 20 |
| Leucine | 1.5 | 1.0 | 31 |
| Lysine | 1.1 | 1.0 | 12 |
| Methionine | 1.6 | 0.9 | 46 |
| Phenylalanine | 1.4 | 1.0 | 24 |
| Proline | 2.0 | 1.5 | 24 |
| Serine | 1.4 | 1.3 | 2 |
| Threonine | 1.9 | 2.0 | −7 |
| Tryptophan | 1.6 | 1.4 | 16 |
| Tyrosine | 1.8 | 1.4 | 24 |
| Valine | 1.9 | 1.2 | 36 |

### Example 6: Enzymes and Probiotics Blend Taken Alone with the Plant Derived Protein (Pea) Increases the Bioavailability of the Amino Acids in the Blood

Aim

Purpose of the study was to evaluate the effect of enzymes and probiotics blend taken along with pea protein on the subsequent appearance of amino acids 5 in the blood.

Study Design and Sample Analysis

Study design is same as explained in example 1. On day 15 of the study, site visit was planned. On arrival to site, after the baseline assessments, subjects were administrated with respective supplements on empty stomach and blood samples were withdrawn at 0 h, 0.5 h, 1 h, 2 h, 3 h and 4 h for amino acid analysis. Amino acids were analyzed using liquid chromatography tandem mass spectrometry method.

Results Variables

Amount of amino acids (i.e. alanine, arginine, asparagine, aspartic acid, cysteine, glutamine, glutamic acid, glycine, histidine, isoleucine, leucine, lysine, methionine, 15 phenylalanine, proline, serine, threonine, tryptophan, tyrosine and valine) in peripheral blood were described by means of: area under the curve (AUC), maximum concentration (Cmax), the corresponding time (Tmax) and, and rate of increase in percentage as well as μmol/min, calculated as appropriate.

The area under the curve (concentration vs. time, in short AUC) was calculated for each of the 20 amino acids through the linear trapezoidal rule and using all available time points. Cmax was defined as the highest observed concentration and Tmax was the time when Cmax was reached. Increase (%) in AUC, increase (%) Cmax and decrease (%) in Tmax was calculated for test group compare to placebo.

Results

Results revealed that the individuals who took pea protein plus enzymes-probiotics blend (test arm) showed increase in AUC and Cmax of alanine (AUC 16.4%, Cmax 19.7%), arginine (AUC 8.8%, Cmax 8.6%), glutamic acid (AUC 6.4%, Cmax 4%), glycine (AUC 5%, Cmax 6.5%), isoleucine (AUC 12.7%, Cmax 15.8%), leucine (AUC 10.8%, Cmax 16.8%), lysine (AUC 11.4%, Cmax 14.1%), methionine (AUC 10.5%, Cmax 16.9%), phenylalanine (AUC 14.7%, Cmax 14.9%), proline (AUC 15.1%, Cmax 22.3%), serine (AUC 14%, Cmax 17.3%), threonine (AUC 15.2%, 5 Cmax 19.1%), tryptophan (AUC 9.9%, Cmax 13.9%), tyrosin (AUC 10.3%, Cmax 10.3%) and valine (AUC 7.3%, Cmax 6.5%) compared to the placebo arm.

Also decrease (%) in Tmax was observed for alanine (40%), arginine (18%), asparagine (22%), aspartic acid (19%), cysteine (14%), glutamine (4.8%), glutamic acid (20%), 10 glycine (40%), isoleucine (20%), leucine (22%), lysine (20%), phenylalanine (13%), proline (28%), serine (17%), methionine (20%), threonine (22%), tryptophan (6.1%), tyrosine (23%) and valine (7.1%).

Results indicated that the rate of absorption of plasma amino acid concentration (μmol/min) in test arm was increased compared to that of in the placebo arm. In particular, arginine (25%), glutamine (68%), histidine (4.2%), isoleucine (25%), leucine (27%), lysine (26%), methionine (41%), phenylalanine (7%), serine (35%), threonine (57%), tryptophan (44%), tyrosine (41%) and valine (5.4%), which includes both essential amino acids and branched chain amino acids. Oral consumption of the pea protein along with proteolytic enzymes and at least one bacterial strain from Bacillus coagulans, Bacillus clausii, Bacillus subtilis, Lactobacillus acidophilus and Lactobacillus plantarum increases the blood bioavailability of amino acids derived from plant proteins. Given protein plus enzymes-probiotics blend not only increases the plasma amino acid concentration but also decreases its Tmax.

Tables

Tables 10-12 reports the results for the participant (n=15) consuming plant origin protein (Pea) with and without enzymes-probiotics blend in a randomized, cross-over double blind clinical trial.

Table 10 reports the rate of absorption of amino acid in the subjects consuming pea protein and the subjects consuming pea protein with enzymes-probiotics blend.

Table 11 reports the AUC and Cₘₐₓ of amino acid in the subjects consuming pea protein and the subjects consuming pea protein with enzymes-probiotics blend.

Table 12 reports Tₘₐₓ of amino acid in the subjects consuming pea protein and the subjects consuming pea protein with enzymes-probiotics blend.

TABLE 10
Rate of absorption of plasma amino acids in the subjects
consuming pea protein and the subjects consuming
pea protein with enzymes-probiotics blend.

| | Rate of increase in plasma amino acid | | Increase in |
|---|---|---|---|
| | Concentration (μmol/min) | | plasma |
| | Whey | Whey Protein + Enzymes- | amino acid |
| Amino acids | Protein | probiotics blend | (%) |
| Alanine | 2.05 | 1.85 | −9.9 |
| Arginine | 0.64 | 0.81 | 25 |
| Asparagine | 1.07 | 1.03 | −3.7 |
| Asparatic acid | 0.003 | 0 | −8 |
| Cysteine | 0.16 | 0.1 | −33 |
| Glutamic acid | 0.35 | 0.27 | −23 |
| Glutamine | 1.59 | 2.67 | 68 |
| Glycine | 1.26 | 0.87 | −30 |
| Histidine | 0.47 | 0.49 | 4.2 |
| Isoleucine | 1.32 | 1.65 | 25 |
| Leucine | 2.08 | 2.65 | 27 |
| Lysine | 1.84 | 2.32 | 26 |
| Methionine | 0.1 | 0.13 | 41 |
| Phenylalanine | 0.42 | 0.45 | 7 |
| Proline | 1.02 | 0.39 | −62 |
| Serine | 0.58 | 0.79 | 35 |
| Threonine | 0.7 | 1.1 | 57 |
| Tryptophan | 0.19 | 0.27 | 44 |
| Tyrosine | 0.61 | 0.86 | 41 |
| Valine | 1.75 | 1.85 | 5.4 |

TABLE 11
AUC and Cₘₐₓ of plasma amino acid in the subjects
consuming pea protein and the subjects consuming
pea protein with enzymes-probiotics blend.

| | | | Pea Protein + | | | |
|---|---|---|---|---|---|---|
| | | | Enzymes-probiotics | | | |
| Amino | Pea Protein | | blend | | Increase (%) | |
| acids | AUC | Cₘₐₓ | AUC | Cₘₐₓ | AUC | Cₘₐₓ |
| Alanine | 2456.6 | 711.8 | 2860.1 | 851.9 | 16.4 | 19.7 |
| Arginine | 305.4 | 101.5 | 332.2 | 110.3 | 8.8 | 8.6 |
| Asparagine | 505.3 | 162.4 | 505.2 | 167.2 | 0.0 | 3.0 |
| Aspartic | 4.0 | 1.2 | 3.8 | 1.2 | −5.7 | −3.6 |
| acid | | | | | | |
| Cystine | 163.0 | 54.1 | 124.5 | 42.6 | −23.6 | −21.3 |
| Glutamine | 4202.0 | 1193.8 | 3810.0 | 1101.4 | −9.3 | −7.7 |
| Glutamic | 208.6 | 72.4 | 221.9 | 75.3 | 6.4 | 4.0 |
| acid | | | | | | |
| Glycine | 1621.3 | 469.2 | 1702.5 | 499.6 | 5.0 | 6.5 |
| Histidine | 359.4 | 117.2 | 340.0 | 110.2 | −5.4 | −6.0 |
| Isoleucine | 505.2 | 164.3 | 569.2 | 190.2 | 12.7 | 15.8 |
| Leucine | 824.1 | 274.5 | 913.4 | 320.6 | 10.8 | 16.8 |
| Lysine | 706.9 | 239.4 | 787.8 | 273.3 | 11.4 | 14.1 |
| Methionine | 76.9 | 24.7 | 85.0 | 28.9 | 10.5 | 16.9 |
| Phenylalnine | 244.1 | 74.6 | 279.9 | 85.7 | 14.7 | 14.9 |
| Proline | 1157.1 | 333.2 | 1331.3 | 407.5 | 15.1 | 22.3 |
| Serine | 516.9 | 158.7 | 589.4 | 186.2 | 14.0 | 17.3 |
| Threonine | 593.0 | 179.1 | 682.9 | 213.3 | 15.2 | 19.1 |
| Tryptophan | 226.8 | 65.4 | 249.1 | 74.6 | 9.9 | 13.9 |
| Tyrosine | 391.4 | 120.3 | 431.9 | 132.7 | 10.3 | 10.3 |
| Valine | 1003.6 | 302.1 | 1077.3 | 321.8 | 7.3 | 6.5 |

TABLE 12
Tₘₐₓ of plasma amino acid in the subjects consuming
pea protein and the subjects consuming pea
protein with enzymes-probiotics blend.

| | | Pea Protein + | |
|---|---|---|---|
| | Pea | Enzymes-probiotics | Decrease |
| Amino acid | Protein | blend | (%) |
| Alanine | 1.8 | 1.1 | 40 |
| Arginine | 1.7 | 1.4 | 18 |
| Asparagine | 1.5 | 1.2 | 22 |
| Aspartic acid | 1.9 | 1.6 | 19 |
| Cysteine | 1.7 | 1.5 | 14 |
| Glutamine | 1.4 | 1.3 | 4.8 |
| Glutamic acid | 1.8 | 1.5 | 20 |
| Glycine | 1.4 | 0.9 | 40 |
| Histidine | 1.3 | 1.6 | −24 |
| Isoleucine | 1.7 | 1.3 | 20 |
| Leucine | 1.7 | 1.3 | 22 |
| Lysine | 1.5 | 1.2 | 20 |
| Methionine | 1.3 | 1.1 | 20 |
| Phenylalanine | 1.8 | 1.6 | 13 |
| Proline | 1.6 | 1.1 | 28 |
| Serine | 1.6 | 1.3 | 17 |
| Threonine | 1.5 | 1.2 | 22 |
| Tryptophan | 1.1 | 1.0 | 6.1 |
| Tyrosine | 2.0 | 1.5 | 23 |
| Valine | 1.9 | 1.7 | 7.1 |

The foregoing description of specific embodiments of the present invention has been presented for purposes of illustration and description. They are not intended to be exhaustive or to limit the present invention to the precise forms disclosed, and obviously many modifications and variations are possible in light of the above teaching.

The embodiments were chosen and described to best explain the principles of the present invention and its practical application, to thereby enable others, skilled in the art to best utilize the present invention and various embodiments with various modifications as are suited to the particular use contemplated.

It is understood that various omission and substitutions of equivalents are contemplated as circumstance may suggest or render expedient, but such are intended to cover the application or implementation without departing from the scope of the present invention.

What is claimed is:

1. A method for preventing negative effects of protein indigestion on gut microbiota, promoting microflora balance and increasing bioavailability of postbiotics, the method comprising administration of protein or protein derivatives, enzymes, probiotics and optionally one or more other components.

2. The method of claim 1, wherein protein indigestion occurs due to excess protein intake, a resistance of the consumed protein to digestion, a digestive enzyme deficiency, age-related digestive inefficiency, or other impediment to protein digestion.

3. The method of claim 1, wherein the protein or the protein derivative is derived from plants, animals or microbes.

4. The method of claim 1, wherein the gut microbiota has more diverse strains and an increase in the amount of beneficial bacteria.

5. The method of claim 1, wherein the probiotic includes: Bacillus coagulans, Bacillus clausii, Bacillus subtilis, Lactobacillus acidophilus and/or Lactobacillus plantarum.

6. The method of claim 1, wherein the enzymes include one or more proteases including endoproteases, exoproteases, carboxyproteases, fungal proteases, bacterial proteases, peptidases, amino proteases and mixtures thereof.

7. The method of claim 1, further comprising taking the enzymes and the protein or the protein derivatives simultaneously or serially wherein the enzymes are taken within 3 hours after the protein or the protein derivatives are consumed or the enzymes are taken within 1 hour prior to the protein consumption, and the probiotics are taken at any point within the same day.

8. The method of claim 1, wherein the increased bioavailability of the postbiotics is measured by a number of metrics including absorption rate, AUC, Cmax and/or Tmax.

9. The method of claim 1, wherein the postbiotics include gamma amino butyric acid (GABA), L-citrulline, β-alanine, L-ornithine and/or anserine.

10. The method of claim 1, further comprising increasing a bioavailability of gamma amino butyric acid (GABA), the GABA modulating synaptic transmission, promoting neuronal development and relaxation, preventing sleeplessness and depression, protecting the cardiovascular system, providing functional antioxidant management pain relief, preventing diabetes, and/or treating depression, insomnia, cognitive impairment, memory loss, mood disorders, seizures, and/or epilepsy.

11. The method of claim 1, further comprising increasing a bioavailability of L-citrulline, the L-citrulline reducing blood pressure, regulating of innate and adaptive immunity, providing functional antioxidant management, promoting muscle and metabolic health, promoting cardiac function, reducing age-related muscle damage, regulating endothelial vasodilation, and/or promoting anti-diabetic functions.

12. The method of claim 1, further comprising increasing a bioavailability of β-alanine, the β-alanine improving athletic performance, enhancing cognitive function during stressful conditions, improving resiliency to stress, and/or increasing cellular oxygen consumption and an expression of cellular proteins associated with oxidative metabolism.

13. The method of claim 1, further comprising increasing a bioavailability of L-ornithine, the L-ornithine reducing stress, improving sleep quality, reducing physical fatigue, increasing mental ability, improving skin aesthetics, promoting synthesis and production of collagen, increasing muscle growth and/or preventing obesity.

14. The method of claim 1, further comprising increasing a bioavailability of anserine, in the anserine relieving fatigue and/or stress, modulating anxiety, modulating post-partum lactation, improving physical performance, improving immunity, reducing hyperglycemia and/or hypertension, enhancing immunity, preventing age related neurological disorders, enhancing defense against infectious diseases and/or accelerating wound healing.

15. The method of claim 1, wherein the administering step includes administering a serving of a blend of probiotics including 0.05 billion to 100 billion CFU of Bacillus coagulans, 0.05 billion to 100 billion CFU of Bacillus clausii, 0.05 billion to 100 billion CFU of Bacillus subtilis, 0.05 billion to 100 billion CFU of Lactobacillus acidophilus, and 0.05 billion to 100 billion CFU of Lactobacillus plantarum.

16. The method of claim 1, wherein the enzymes comprise 1 mg to 5 g in each serving of the components administered.

17. The method of claim 1, wherein a combination of the probiotics and enzymes are consumed at a dose of 0.01 to 10% of the weight of the protein or protein derivatives.

18. A method for increasing absorption of amino acids, the method comprising administering a protein or protein derivatives, enzymes and probiotics, wherein the enzymes are selected from one or more of endoproteases, exproteases, carboxyproteases, fungal proteases, bacterial proteases, peptidases, and amino proteases, and the probiotics are selected from one or more of Bacillus coagulans, Bacillus clausii, Bacillus subtilis, Lactobacillus acidophilus and Lactobacillus plantarum.

19. The method of claim 18, wherein the method increases the bioavailability of the amino acids, the amino acids including essential amino acids, non-essential amino acids, branched-chain amino acids or a mixture thereof.
